(19) United States
(12) Patent Application Publication
(10) Pub. No.: US 2025/0031975 A1
(43) Pub. Date: Jan. 30, 2025

(54) TEMPERATURE MEASURING METHOD AND APPARATUS BASED ON CREATINE CHEMICAL EXCHANGE SATURATION TRANSFER IMAGING

(71) Applicant: SHENZHEN INSTITUTES OF ADVANCED TECHNOLOGY, Nanshan Shenzhen (CN)

(72) Inventors: Lijuan Zhang, Shenzhen (CN); Chao Zou, Shenzhen (CN); Siqi Cai, Shenzhen (CN); Shihui Zhou, Shenzhen (CN); Yang Zhou, Shenzhen (CN); Hairong Zheng, Shenzhen (CN); Xin Liu, Shenzhen (CN)

(73) Assignee: SHENZHEN INSTITUTES OF ADVANCED TECHNOLOGY, Nanshan Shenzhen (CN)

(21) Appl. No.: 18/836,155

(22) PCT Filed: Jan. 19, 2022
(86) PCT No.: PCT/CN2022/072719

§ 371 Date: Aug. 6, 2024

(30) Foreign Application Priority Data
May 18, 2021 (CN) 202110540092.2
Jan. 10, 2022 (CN) 202210022362.5

(51) Int. Cl. A61B 5/01; G01K 11/00

(52) U.S. Cl. CPC A61B 5/01; G01K 11/00

(57) ABSTRACT

A temperature measuring method and apparatus based on creatine chemical exchange saturation transfer (CEST) imaging. The method comprises the following steps: (1) performing creatine CEST imaging on a creatine phantom, and analyzing a chemical shift of creatine relative to water in the creatine phantom; (2) fitting a mathematical relation between the chemical shift of the creatine relative to water and the temperature; and (3) performing CEST imaging on creatine in a sample, and calculating the temperature according to the mathematical relation, fitted in step (2), between the chemical shift of the creatine relative to water and the temperature. In the temperature measuring method, the creatine is taken as an endogenous reference, and highly-spatial-resolution, highly-sensitive, and non-invasive absolute temperature measurement can be implemented by means of temperature dependence of a CEST effect of Cr and water.

### TECHNICAL FIELD

The present application belongs to the technical field of noninvasive temperature measurement, and relates to a temperature-measuring method and a device based on creatine chemical exchange saturation transfer imaging.

### BACKGROUND

The brain tissue temperature can fluctuate with neural activity and brain metabolism, and is also regulated and influenced by body temperature through blood circulation. The brain tissue temperature is a comprehensive indicator reflecting physiological characteristics of the tissue such as substance metabolism, tissue perfusion, and vascular autoregulation ability. Most of the physicochemical reactions involved in the cerebral neuronal activity are temperature-sensitive, and many diseases (such as cerebral trauma, stroke, tumor, multiple sclerosis, and epilepsy) can disrupt the homeostasis of brain temperature, resulting in local brain temperature abnormality and changes in the spatial distribution pattern of brain temperature, and causing a series of responses such as abnormal cellular metabolism, secondary neuronal damage, and damage to blood vessels and blood-brain barrier. Therefore, noninvasive absolute temperature mapping technology is of great significance in investigating the brain temperature regulation mechanisms under physiological and pathological conditions and deeply investigating the complex pathological mechanisms of brain injury.

Magnetic resonance thermometry method is mainly based on the temperature dependence of magnetic resonance parameters including proton density, T1 and T2 relaxation times, diffusion coefficient, proton resonance frequency, and magnetization transfer, etc. Currently, the relatively widely used absolute thermometry techniques based on magnetic resonance mainly include: (1) based on the temperature dependence of resonance frequency of water hydrogen proton 1H and the temperature insensitivity of the chemical shift of some macromolecular substances, for example, N-acetyl-aspartic acid (NAA), the chemical shift of reference macromolecular substances relative to water hydrogen protons is measured at different temperatures by magnetic resonance spectroscopy (MRS) imaging technology, and the relationship between the chemical shift of the reference substance and temperature is fitted, achieving the noninvasive thermometry based on the proton resonance frequency (PRF); however, the imaging resolution of this method is relatively low, and the temperature measurement is susceptible to motion and magnetic field drift; (2) the absolute temperature of cerebrospinal fluid or other tissues can be measured based on the relationship between the free diffusion coefficient of water molecules and the temperature, but at present this technique is only applicable to detect the temperature of pure water tissues, and cannot be applied to tissues where the diffusion of water molecules is restricted; (3) a study uses a paramagnetic chelate complex as the exogenous reference substance by injection, measures the chemical shift of chelate complex at different temperatures based on chemical exchange saturation transfer (CEST) imaging technology, and fits the linear relationship between the chemical shift and temperature (refers to: Zhang S, Malloy C R, AD Sherry. MRI thermometry based on PARACEST agents [J]. Journal of the American Chemical Society, 2005, 127 (50): 17572); however, the biological safety of paramagnetic chelate complex needs to be considered, and the paramagnetic chelate complex is not conducive to clinical promotion because the repetitive administration of injections for thermometry is less practical.

In summary, it is of great significance to develop an accurate and noninvasive method for brain thermometry for exploring the mechanisms of brain temperature regulation under physiological and pathological conditions.

### SUMMARY

The present application provides a temperature-measuring method and a device based on creatine chemical exchange saturation transfer imaging. The method is based on the relationship between the chemical exchange saturation transfer effect of creatine and the temperature, in combination with magnetic resonance imaging technology to achieve noninvasive absolute temperature measurement with high spatial resolution and high sensitivity.

In a first aspect, the present application provides a temperature-measuring method based on creatine chemical exchange saturation transfer imaging, and the method comprises the following steps:
(1) performing chemical exchange saturation transfer imaging on a creatine phantom, and analyzing the chemical shift of creatine in the creatine phantom relative to water;
(2) performing fitting to model a mathematical relationship between the chemical shift of creatine relative to water and creatine phantom temperatures; and
(3) performing chemical exchange saturation transfer imaging on creatine in a sample, analyzing the chemical shift of creatine in the sample relative to water, and calculating the sample temperature according to the mathematical relationship between the chemical shift of creatine relative to water and the creatine phantom temperature in step (2).

In the present application, creatine (Cr), as an important energy metabolite, has a stable concentration in the brain, and the creatine chemical exchange saturation transfer (Cr-CEST) effect is insensitive to the non-temperature environmental factors such as pH, and its CEST exchange rate under physiological temperature and pH conditions is approximately 7-8 times higher than that of phosphocreatin (PCr). Therefore, creatine is used as an endogenous agent in the present application, and the temperature dependence of CEST effect of creatine and water is used to perform noninvasive absolute temperature measurement with high spatial resolution and high sensitivity.

The temperature-measurement method based on creatine chemical exchange saturation transfer imaging of the present application can be used for a non-disease diagnostic or non-therapeutic purpose, or for scientific research.

Preferably, raw materials for preparing the creatine phantom comprise creatine, agar powder, phosphate buffer, and deionized water.

In the present application, the addition of agar powder can improve the signal strength of the creatine phantom and avoid artifacts caused by heat conduction during the detection.

Preferably, a concentration of creatine in the creatine phantom is 10-120 mmol/L, including but not limited to 11 mmol/L, 12 mmol/L, 13 mmol/L, 15 mmol/L, 20 mmol/L, 30 mmol/L, 40 mmol/L, 50 mmol/L, 60 mmol/L, 70 mmol/L, 80 mmol/L, 90 mmol/L, 100 mmol/L, 105 mmol/L, 110 mmol/L, 112 mmol/L, 115 mmol/L, 118 mmol/L, or 119 mmol/L.

Preferably, a reagent purity of the creatine is greater than 98%.

Preferably, a pH of the creatine phantom is 6.0-7.2, including but not limited to 6.1, 6.2, 6.3, 6.4, 6.6, 6.7, 6.8, 6.9, or 7.1.

Preferably, a temperature-measuring range of the creatine phantom is 10-43° C., including but not limited to 11° C., 12° C., 13° C., 14° C., 15° C., 16° C., 17° C., 18° C., 19° C., 20° C., 25° C., 30° C., 35° C., 36° C., 37° C., 38° C., or 39° C.

Preferably, the temperature measurement based on creatine chemical exchange saturation transfer imaging further comprises a step of preparing the creatine phantom.

Preferably, a method for preparing the creatine phantom comprises:
creatine, agar powder, phosphate buffer, and deionized water are mixed and heated, and the pH is adjusted to obtain the creatine phantom; specifically, the creatine, agar powder, phosphate buffer, and deionized water are mixed to obtain creatine phantom solutions with different concentrations, and the solution pH is adjusted by dropwise adding sodium hydroxide solution or hydrochloric acid solution, and the resulting mixed solutions are heated until the agar powder is completely dissolved, stirred uniformly and then added in plastic containers to naturally cool down.

Preferably, the chemical exchange saturation transfer imaging is performed on the creatine phantom in step (1), a constant-temperature water bath is set to a specific temperature, creatine phantoms prepared with different pH and different creatine concentrations are placed into the water bath, a probe of optical fiber thermometer is inserted to monitor the real-time temperature of the creatine phantom, and after the phantom temperature is stable, the CEST scan is started; firstly, the excitation pulse sequence without pre-saturation is used in scan to obtain an image as a reference, and the signal intensity is denoted as S₀; then, a series of pre-saturation excitation pulse sequences with different offset frequencies Δω are used in scan to obtain a series of images, and the signal intensity is denoted as Sₛₐₜ(Δω); the signal proportion Sₛₐₜ(Δω)/S₀ represents the signal attenuation under the action of pre-saturation excitation pulse with the offset frequency of Δω, which is defined as z-spectra. When the creatine phantom is excited and saturated by the pre-saturation excitation pulse at frequency Δω, the saturation effect can be transferred to the water hydrogen protons via the dynamic exchange between the creatine amine protons with the water hydrogen protons, leading to a reduction in the magnetic resonance signal of the water hydrogen protons. When the frequency of pre-saturation excitation pulse is equal to the resonance frequency of water hydrogen protons, all the water hydrogen protons are saturated, and the acquisition signal tends towards zero. As the offset frequency increases, the signal of saturated water gradually decreases, while the signal of magnetic resonance gradually increases. When the offset frequency is exactly equal to the excitation frequency of creatine amine protons, the creatine amine protons are saturated and exchanged with the water hydrogen protons, and the acquisition signal intensity corresponding to this frequency exhibits a decreasing trend.

The data Sₛₐₜ(+Δω) and Sₛₐₜ(−Δω) which are symmetric relative to the resonance frequency of water hydrogen protons on the z-spectrum are subjected to subtraction and then divided by Sₛₐₜ(−Δω), as shown in Equation (1); the result characterizes the asymmetry of each point on the proton resonance frequency curve, denoted as CEST_{asym}. The frequency offset corresponding to the maximum CEST_{asym} is the chemical shift of creatine amine protons.

$${CEST}_{asym} = \frac{{S}_{sat} ( - Δω ) - {S}_{sat} ( + Δω )}{{S}_{sat} ( - Δω )} . \qquad ( 1 )$$

The temperature of the constant-temperature water bath is changed, and after the creatine phantom temperature is stable, the above process is repeated, and the chemical shifts of the creatine amine protons measured at different temperatures are recorded.

Preferably, a mathematical relationship between the chemical shift of creatine relative to water and the creatine phantom temperature is fitted; the resulting mathematical relationship is shown in Equation (2):

$$T ( ° C . ) = 126.821 \times Δω - 220.811 , \qquad ( 2 )$$

wherein Δω represents the chemical shift (ppm) of creatine amine protons relative to water, and the R² is 0.893, and the p value is less than 0.001.

In addition, the z-spectra of the water hydrogen protons and creatine amine protons can be modeled by the multi-pool Lorentzian fitting, as shown in Equation (3), to improve the accuracy of calculating the chemical shift of creatine amine protons.

$$1 - \frac{{S}_{sat} ( Δω )}{{S}_{0}} = {\sum }_{i = 1}^{N} \frac{{A}_{i}}{1 + 4 {( \frac{Δω - {ω}_{i}}{{σ}_{i}} )}^{2}} , \qquad ( 3 )$$

wherein Aᵢ, ωᵢ, and σᵢ represent the amplitude, chemical shift, and linewidth of the z spectrum for the proton pool labeled number i, respectively, and N represents the total number of proton pools.

According to the calculation result of Equation (3), a mathematical relationship between the chemical shift of creatine relative to water and the creatine phantom temperature is fitted, and the obtained mathematical relationship is shown in Equation (4):

$$T ( ° C . ) = 169.519 \times Δω - 302.907 , \qquad ( 4 )$$

wherein Δω represents the chemical shift (ppm) of creatine amine protons relative to water, and the R² is 0.956, and the p value is less than 0.001; this equation has a high fitting accuracy.

In the present application, a method for the chemical exchange saturation transfer imaging comprises performing signal acquisition by a pre-saturation excitation pulse combined with a spin echo-echo planar sequence or a gradient echo sequence, and performing interval imaging.

Preferably, the pre-saturation excitation pulse comprises 10 rectangular pulses.

Preferably, a duration of the rectangular pulse is 90-110 ms (such as 91 ms, 92 ms, 93 ms, 95 ms, 98 ms, 100 ms, 105 ms, 106 ms, 108 ms, or 109 ms), and B1=0.1-0.3 μT.

Preferably, a method for the interval imaging comprises performing interval imaging via pre-saturation excitation pulses with the resonance frequency offset ranging from −3.0 ppm to +3.0 ppm relative to the resonance frequency of water hydrogen protons, with more than 200 intervals.

As a preferred technical solution, the technology roadmap of the temperature-measuring method based on creatine chemical exchange saturation transfer imaging is shown in FIG. 1, which specifically comprises the following steps:

(1) Creatine, agar powder, phosphate buffer, and deionized water are mixed and heated, and the pH is adjusted to 6.0-7.2, wherein the concentration of creatine is 10-120 mmol/L, so as to obtain the creatine phantom.

(2) Chemical exchange saturation transfer imaging is performed on the creatine phantom; wherein the CEST sequence consists of two parts: a pre-saturation excitation pulse and an image signal acquisition sequence, in a 3.0 T magnetic resonance system, the pre-saturation excitation pulse consists of ten rectangular pulses, each rectangular pulse has a duration of 90-110 ms, and B1=0.1-0.3 μT, signal acquisition is performed by combining a spin echo-echo planar sequence or a gradient echo sequence, and interval imaging is performed via pre-saturation excitation pulses with the resonance frequency offset ranging from −3.0 ppm to +3.0 ppm relative to the resonance frequency of water hydrogen protons, with more than 200 intervals. Firstly, the excitation pulse sequence without pre-saturation is used in scan to obtain an image as a reference, and the signal intensity is denoted as S₀; then, a series of pre-saturation excitation pulse sequences with different frequency offset Δω are used in scan to obtain a series of images, and the signal intensity is denoted as Sₛₐₜ(Δω). The signal proportion Sₛₐₜ(Δω)/S₀ represents the signal attenuation under the action of pre-saturation excitation pulse with the frequency offset of Δω, which is defined as z-spectra. The data Sₛₐₜ(+Δω) and Sₛₐₜ(−Δω) which are symmetric about the resonance frequency of water hydrogen protons on the z-spectra are subjected to subtraction and then divided by Sₛₐₜ(−Δω), and the result characterizes the asymmetry of each point on the proton resonance frequency curve, denoted as CEST_{asym}. The frequency offset corresponding to the maximum CEST_{asym} is the chemical shift of creatine amine protons relative to water. Moreover, the multi-pool Lorentzian fitting is optionally used to model the z-spectra of proton pools, thereby obtaining the chemical shift of creatine amine protons relative to water.

(3) A mathematical relationship between the chemical shift of creatine relative to water and the creatine phantom temperature is modeled by fitting; the relationship between the chemical shift of creatine relative to water and the creatine phantom temperature is modeled according to the chemical shift of creatine amine protons relative to water measured at different temperatures and the corresponding phantom temperatures recorded by a probe of an optical fiber thermometer.

The above process can be repeatedly carried out on creatine phantoms with different concentrations and pH values, and the relationship between the chemical shift of creatine relative to water and the temperature can be re-fitted to evaluate the relationship between the chemical shift of creatine relative to water and the temperature in reliability, repeatability, and stability insusceptible to other environmental factors.

(4) Chemical exchange saturation transfer imaging is performed on creatine in a sample, the chemical shift of creatine in the sample relative to water is analyzed, and the sample temperature is calculated according to the mathematical relationship between the chemical shift of creatine relative to water and the creatine phantom temperature in step (3).

In a second aspect, the present application provides a temperature-measuring device based on creatine chemical exchange saturation transfer imaging; the temperature-measuring device is used in the temperature-measuring method based on creatine chemical exchange saturation transfer imaging described in the first aspect, and comprises a creatine phantom testing unit, a fitting unit, and a sample testing unit.

The creatine phantom testing unit is used for performing chemical exchange saturation transfer imaging on a creatine phantom and analyzing the chemical shift of creatine in the creatine phantom relative to water; the fitting unit is used for performing fitting to model a mathematical relationship between the chemical shift of creatine relative to water and the creatine phantom temperature; the sample testing unit is used for performing chemical exchange saturation transfer imaging on creatine in a sample, analyzing the chemical shift of creatine in the sample relative to water, and calculating the sample temperature according to the mathematical relationship between the chemical shift of creatine relative to water and the creatine phantom temperature obtained from the fitting unit.

Preferably, the temperature-measuring device further comprises a creatine phantom preparation unit.

The creatine phantom preparation unit is used for mixing creatine, agar powder, phosphate buffer, and deionized water, heating, and adjusting the pH to obtain a creatine phantom.

Preferably, a method for the chemical exchange saturation transfer imaging in the creatine phantom testing unit comprises performing signal acquisition by a pre-saturation excitation pulse combined with a spin echo-echo planar sequence or a gradient echo sequence, and performing interval imaging.

Preferably, the pre-saturation excitation pulse comprises 10 rectangular pulses.

Preferably, the duration of the rectangular pulse is 90-110 ms, and B1=0.1-0.3 μT.

Preferably, a method for the interval imaging comprises performing interval imaging via a pre-saturation excitation pulses with the frequency offsets ranging from −3.0 ppm to +3.0 ppm relative to a resonance frequency of water hydrogen protons, with more than 200 intervals.

Compared with the prior art, the present application has the following beneficial effects:

(1) the temperature-measuring method based on creatine chemical exchange saturation transfer imaging in the present application uses creatine as an endogenous reference, and uses the temperature dependence of CEST effect of creatine and water, achieving non-invasive absolute temperature measurement with high spatial resolution and high sensitivity, which can be applied to noninvasive and label-free brain temperature measurement; and

(2) the temperature-measuring method based on creatine chemical exchange saturation transfer imaging in the present application is highly stable, accurate, easy to operate, non-radioactive, and conducive to widespread utilization.

### BRIEF DESCRIPTION OF DRAWINGS

FIG. 1 is a technology roadmap of the present application.

FIG. 2 is a scanning image of a creatine phantom (pH=6.0, 100 mmol/L) at 38° C.

FIG. 3 is a z-spectrum of a creatine phantom (pH=6.0, 100 mmol/L) at 38° C.

FIG. 4 is a graph showing the relationship between the temperature and the chemical shift of creatine relative to water, which is obtained from symmetry analyses of resonance frequency spectra.

FIG. 5 is z-spectra of creatine CEST effect of swine brain samples at 28° C. based on the multi-pool Lorentzian fitting method.

FIG. 6 is a graph showing the relationship between the temperature and the chemical shift of creatine relative to water, which is obtained from the multi-pool Lorentzian fitting.

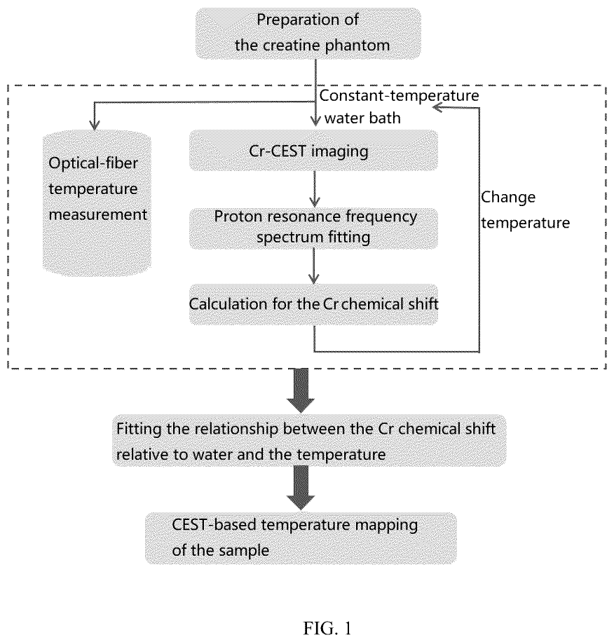

### DETAILED DESCRIPTION

In order to further illustrate the technical methods adopted in the present application and their results, the examples and accompanying drawings are used to further explain the present application hereinafter. It can be understood that the specific embodiments described here are only used to explain the present application, but not to limit the present application.

Examples where the specific technologies or conditions are not indicated can be performed according to the technologies or conditions described in the literature in this field, or the specification of products. The reagents or instruments used without manufacturers indicated are regular products that are commercially available.

### Example 1 Preparation of Creatine Phantom

A creatine monohydrate reagent (C₄H₁₁N₃O₃) (1.4915 g) with the purity greater than 98%, agar powder (1.5 g), phosphate buffer solution (10 mL), and deionized water (90 mL) were prepared into a mixed solution in a volume of 100 mL, with a pH of 6.0 and the creatine concentration of 100 mmol/L; and the solution was heated until the agar powder was completely dissolved, stirred evenly and then added into a plastic tube to naturally cool down, so as to obtain the creatine phantom.

### Example 2 Cr-CEST Experiment

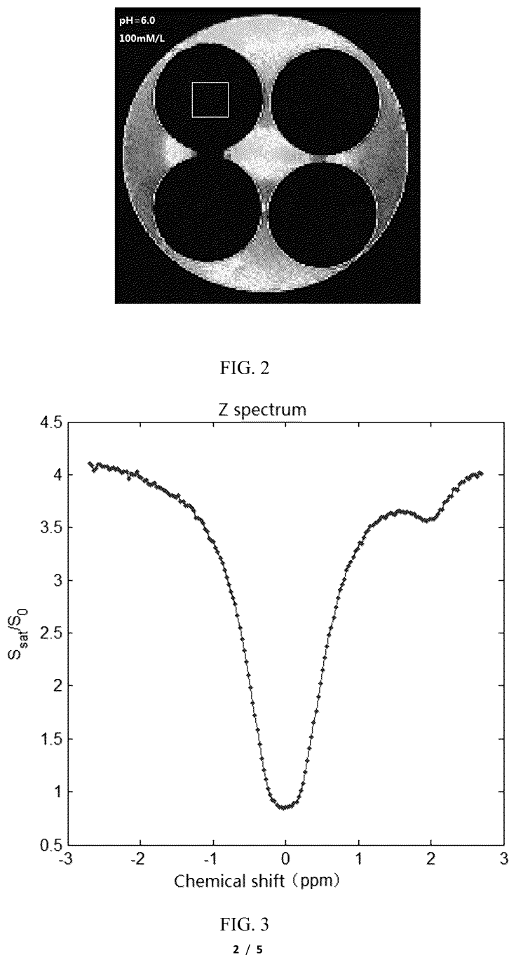

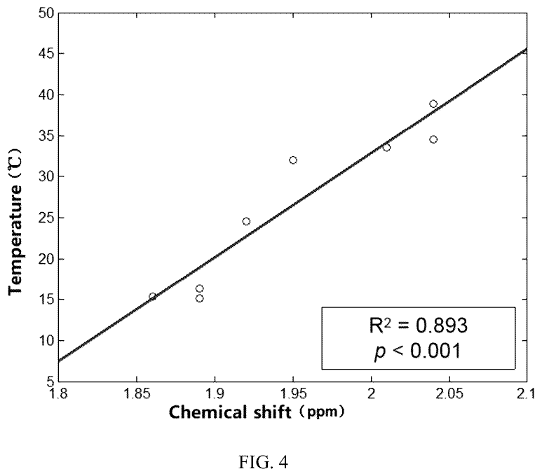

A constant-temperature water bath device was used and set to 38° C. (close to the temperature of human brain); the creatine phantom was placed into the water bath and monitored for the real-time temperature with an optical fiber thermometer; after the temperature was stable, the CEST imaging was carried out (United Imaging uMR 790, 3.0T, 32-channel head-neck coil); the CEST sequence consisted of two parts: a pre-saturation excitation pulse and a spin echo-echo planar imaging sequence; the pre-saturation excitation pulse consisted of ten rectangular pulses, each rectangular pulse had a duration of 100 ms and B1=0.1 or 0.15 μT, the main parameters of the spin echo-echo planar imaging acquisition sequence were TR/TE=4000/38.6 ms, the field of view (FOV) of 80 mm, the slice thickness of 8.0 mm, the FA of 160 degree, the frequency offsets of pre-saturation excitation pulse relative to the resonance frequency of water hydrogen protons were set from −3.0 ppm to +3.0 ppm with intervals of 0.03 ppm and 200 intervals for imaging. Firstly, the excitation pulse sequence without pre-saturation was used in scan to obtain an image as a reference, and the signal intensity was denoted as S₀; then, a series of pre-saturation excitation pulse sequences with different frequency offset Δω were used in scan to obtain a series of images, as shown in FIG. 2, and the signal intensity was denoted as Sₛₐₜ(Δω). The signal proportion Sₛₐₜ(Δω)/S₀ represents the signal attenuation under the action of pre-saturation excitation pulse with the frequency offset of Δω, which is defined as z-spectra. As shown in FIG. 3, the data Sₛₐₜ(+Δω) and Sₛₐₜ(Δω) which are symmetric about the resonance frequency of water hydrogen protons on the z-spectrum are subjected to subtraction and then divided by Sₛₐₜ(Δω), and the result characterizes the asymmetry of each point on the proton resonance frequency curve, denoted as CEST_{asym}. The frequency offset of 2.01 ppm corresponding to the maximum CEST_{asym} is recorded as the chemical shift of creatine amine protons relative to water at this temperature.

By adjusting the temperature of the constant-temperature water bath, based on the symmetry analyses of z-spectra, the chemical shifts of creatine amine protons relative to water at temperatures of 15.1° C., 15.3° C., 16.3° C., 24.5° C., 32.0° C., 33.5° C., 34.5° C., and 38.9° C. were calculated to be 1.89 ppm, 1.86 ppm, 1.89 ppm, 1.92 ppm, 1.95 ppm, 2.01 ppm, 2.04 ppm, and 2.04 ppm, respectively. Besides, based on the multi-pool Lorentzian fitting method, the chemical shifts of creatine amine protons relative to water at temperatures of 13.8° C., 16.30° C., 18.30° C., 20.30° C., 23.56° C., 24.50° C., 29.30° C., 32.00° C., 32.07° C., 33.50° C., 34.50° C., 35.68° C., 38.10° C., and 38.9° C. were fitted to be 1.8770 ppm, 1.8928 ppm, 1.9016 ppm, 1.8946 ppm, 1.9127 ppm, 1.9421 ppm, 1.9615 ppm, 1.9795 ppm, 1.9615 ppm, 1.9804 ppm, 1.9832 ppm, 2.0051 ppm, 2.0263 ppm, and 2.0032 ppm, respectively.

### Example 3 Fitting of Relationship Between Cr-CEST and Temperature

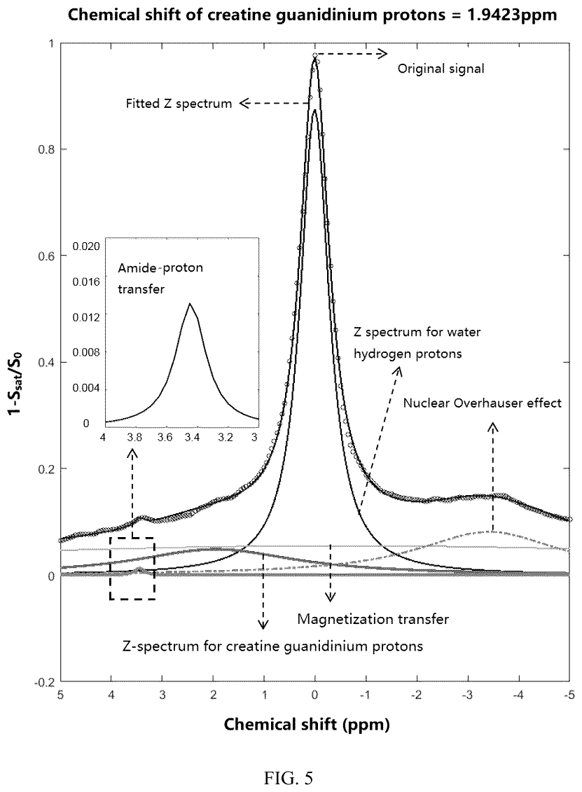

SPSS 19.0 was employed to fit the relationship between temperatures and the chemical shifts of creatine amine protons relative to water that were obtained from the above symmetry analyses of z-spectra and multi-pool Lorentzian fitting method, as shown in FIG. 5; and the resulting mathematical relationship equations are shown by Equations (2) and (4), respectively:

$$T ( ° C . ) = 126.821 \times Δω - 220.811 , \qquad ( 2 )$$

wherein Δω represents the chemical shift (ppm) of creatine amine protons relative to water, and the R² is 0.893, and the p value is less than 0.001.

$$T ( ° C . ) = 169.519 \times Δω - 302.907 , \qquad ( 4 )$$

wherein Δω represents the chemical shift (ppm) of creatine amine protons relative to water, the R² is 0.956, and the p value is less than 0.001; this equation has a higher fitting precision.

### Example 4 Temperature Measurement of Biological Sample Based on Cr-CEST

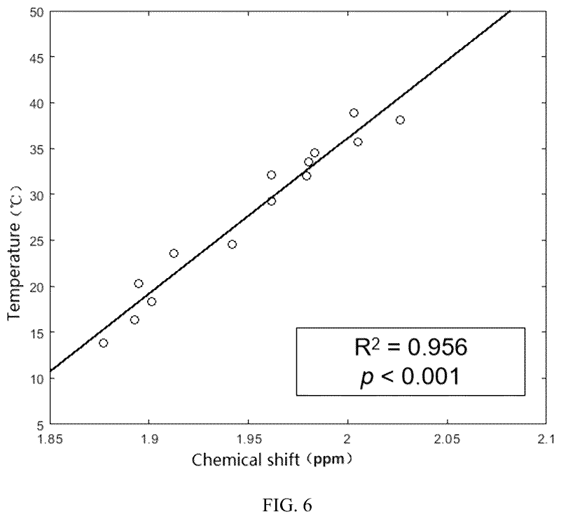

Samples of swine brain tissue homogenate were prepared; and the CEST scanning (Bruker Biospec, 9.4 T) was performed at 28.0° C., with B1=0.23 μT; the frequency offsets of pre-saturation excitation pulse relative to the resonance frequency of water hydrogen protons were set from −5.0 ppm to +5.0 ppm with intervals of 0.05 ppm and total 201 intervals. Based on the multi-pool Lorentzian fitting method (five-pool model: water hydrogen proton pool, creatine amine proton pool, amide proton transfer pool, nuclear Overhauser effect pool, and magnetization transfer pool), the chemical shift of creatine amine protons relative to water at this temperature was fitted to be 1.9423 ppm (FIG. 6); the calculated chemical shift was plugged into Equation (2) to chug a calculated temperature of 25.51° C., and into Equation (4) to chug a calculated temperature of 26.35° C., both of which were close to the actual experiment temperature of 28° C.

Due to the limited available data and numbers of CEST imaging experiments on creatine phantoms in the examples, as well as the relatively simple experimental conditions, the fitting of the relationship between creatine chemical shift and temperature may be affected, which may subsequently impact the accuracy of temperature prediction. In practical applications, the mathematical relationship between Cr-CEST and temperature can be improved in accuracy by methods such as increasing the number of phantom experiments, setting various experimental conditions, optimizing the z-spectrum fitting strategy, and improving the CEST scanning sensitivity.

In conclusion, for the temperature-measuring method based on creatine chemical exchange saturation transfer imaging in the present application, creatine is used as an endogenous agent, and the temperature dependence of the CEST effect of creatine and water is used to perform noninvasive absolute temperature measurement with high spatial resolution and high sensitivity.

The applicant declares that although the detailed method of the present application is described through the above examples, the present application is not limited to the above detailed method, which means that the implementation of the present application does not necessarily depend on the above detailed method. It should be apparent to those skilled in the art that any improvements made to the present application, equivalent substitutions of various raw materials of the product, the addition of adjuvant ingredients, the selection of specific manners, etc., shall all fall within the protection scope and the disclosure scope of the present application.

1. A temperature-measuring method based on creatine chemical exchange saturation transfer imaging, comprising the following steps:
(1) performing chemical exchange saturation transfer imaging on a creatine phantom, and analyzing the chemical shift of creatine in the creatine phantom relative to water;
(2) performing fitting to model a mathematical relationship between the chemical shift of creatine relative to water and a creatine phantom temperature; and
(3) performing chemical exchange saturation transfer imaging on creatine in a sample, analyzing the chemical shift of creatine in the sample relative to water, and calculating a sample temperature according to the mathematical relationship between the chemical shift of creatine relative to water and the creatine phantom temperature in step (2).

2. The temperature-measuring method based on creatine chemical exchange saturation transfer imaging according to claim 1, wherein raw materials for preparing the creatine phantom comprise creatine, agar powder, and phosphate buffer.

3. The temperature-measuring method based on creatine chemical exchange saturation transfer imaging according to claim 1, wherein a concentration of creatine in the creatine phantom is 10-120 mmol/L;
preferably, a reagent purity of the creatine is greater than 98%;
preferably, a pH of the creatine phantom is 6.0-7.2;
preferably, a temperature-measuring range of the creatine phantom is 10-43° C.

4. The temperature-measuring method based on creatine chemical exchange saturation transfer imaging according to claim 1, wherein the method further comprises a step of preparing the creatine phantom;
preferably, a method for preparing the creatine phantom comprises the following steps:
mixing creatine, agar powder, phosphate buffer, and deionized water and heating, and adjusting a pH to obtain the creatine phantom.

5. The temperature-measuring method based on creatine chemical exchange saturation transfer imaging according to claim 1, wherein a method for the chemical exchange saturation transfer imaging comprises performing signal acquisition by a pre-saturation excitation pulse combined with a spin echo-echo planar sequence or a gradient echo sequence, and performing interval imaging;
preferably, the pre-saturation excitation pulse comprises 10 rectangular pulses;
preferably, a duration of the rectangular pulse is 90-110 ms, and B1=0.1-0.3 μT;
preferably, a method for the interval imaging comprises performing interval CEST imaging using a series of the pre-saturation excitation pulses with frequency offsets relative to the resonance frequency of water hydrogen protons ranging from −3.0 ppm to +3.0 ppm, with more than 200 intervals.

6. The temperature-measuring method based on creatine chemical exchange saturation transfer imaging according to claim 1, wherein the method comprises the following steps:
(1) mixing creatine, agar powder, phosphate buffer, and deionized water and heating, and adjusting a pH to 6.0-7.2, wherein a concentration of creatine is 10-120 mmol/L, so as to obtain the creatine phantom;
(2) performing chemical exchange saturation transfer imaging on the creatine phantom, wherein each rectangular pulse in a pre-saturation excitation pulse has a duration of 90-110 ms, and B1=0.1-0.3 μT, signal acquisition is performed by using a spin echo-echo planar sequence or a gradient echo sequence, and interval imaging is performed using a series of the pre-saturation excitation pulses with frequency offsets relative to the resonance frequency of water hydrogen protons ranging from −3.0 ppm to +3.0 ppm, with more than 200 intervals, and analyzing the chemical shift of creatine in the creatine phantom relative to water;
(3) performing fitting to model a mathematical relationship between the chemical shift of creatine relative to water and a creatine phantom temperature; and
(4) performing chemical exchange saturation transfer imaging on creatine in a sample, analyzing the chemical shift of creatine in the sample relative to water, and calculating a sample temperature according to the mathematical relationship between the chemical shift of creatine relative to water and the creatine phantom temperature in step (3).

7. A temperature-measuring device based on creatine chemical exchange saturation transfer imaging, which is used in the temperature-measuring method based on creatine chemical exchange saturation transfer imaging according to claim 1;
the temperature-measuring device comprises a creatine phantom simulation unit, a fitting unit, and a sample testing unit;
the creatine phantom simulation unit is used for performing chemical exchange saturation transfer imaging on a creatine phantom and analyzing the chemical shift of creatine in the creatine phantom relative to water;
the fitting unit is used for performing fitting to model a mathematical relationship between the chemical shift of creatine relative to water and a creatine phantom temperature; and
the sample testing unit is used for performing chemical exchange saturation transfer imaging on creatine in a sample, and calculating a sample temperature according to the mathematical relationship between the chemical shift of creatine relative to water and the creatine phantom temperature obtained from the fitting unit.

8. The temperature-measuring device according to claim 7, wherein the temperature-measuring device further comprises a creatine phantom preparation unit;
the creatine phantom preparation unit is used for mixing creatine, agar powder, phosphate buffer, and deionized water and heating, and adjusting a pH to obtain the creatine phantom.

9. The temperature-measuring device according to claim 7, wherein a method for the chemical exchange saturation transfer imaging in the creatine phantom simulation unit comprises performing signal acquisition by a pre-saturation excitation pulse combined with a spin echo-echo planar sequence or a gradient echo sequence, and performing interval imaging.

10. The temperature-measuring device according to claim 9, wherein the pre-saturation excitation pulse comprises 10 rectangular pulses;
preferably, a duration of the rectangular pulse is 90-110 ms, and B1=0.1-0.3 μT.

11. The temperature-measuring device according to claim 9, wherein a method for the interval imaging comprises performing interval CEST imaging using a series of the pre-saturation excitation pulses with frequency offsets relative to the resonance frequency of water hydrogen protons ranging from −3.0 ppm to +3.0 ppm, with more than 200 intervals.
